(19) United States
(12) Patent Application Publication
(10) Pub. No.: US 2009/0227847 A1
(43) Pub. Date: Sep. 10, 2009

(54) Tunable Light Controller

(76) Inventors: John Tepper, Carrollton, TX (US); Austin Crowder, Dallas, TX (US)

Correspondence Address: DUNLAP CODDING, P.C., PO BOX 16370, OKLAHOMA CITY, OK, 73113, US

(21) Appl. No.: 12/400,486

(22) Filed: Mar. 9, 2009

Related U.S. Application Data
(60) Provisional application No. 61/034,674, filed on Mar. 7, 2008.

(51) Int. Cl. G05D 25/00; A61B 17/00

(52) U.S. Cl. 600/249

(57) ABSTRACT

A surgical illumination device for illuminating a surgical field to provide enhanced visual perception of a tissue during a medical procedure. The surgical illumination device includes a surgical light and a tunable light controller. The surgical light includes a first light source providing a first wavelength of light, a second light source providing a second wavelength of light, and a combiner receiving and combining the first and second wavelengths of light. The tunable light controller includes a database and a tuning device. The database is organized by medical procedure and stores an identification of the medical procedure and at least one pre-programmed color setting for each medical procedure. The preprogrammed color setting is adapted to facilitate a first assigned illumination. The tuning device communicates with the database. The tuning device retrieves the pre-programmed color setting from the database and controls the first light source and the second light source such that the first wavelength and the second wavelength combine to provide the first assigned illumination.

### CROSS-REFERENCE TO RELATED APPLICATIONS

The present patent application claims priority to the provisional patent application identified by U.S. Ser. No. 61/034,674 filed on Mar. 7, 2008, the entire content of which is hereby incorporated herein by reference.

### STATEMENT REGARDING FEDERALLY SPONSORED RESEARCH OR DEVELOPMENT

Not applicable.

### BACKGROUND OF THE INVENTION

There are a number of common open surgical procedures within the medical field including cardiothoracic, neurosurgery, orthopedic surgery, and others. Such procedures call for the surgeon's ability to identify and distinguish different tissues and anatomical structures. It becomes critical that the surgeon have clear vision in order to perform the required tasks.

A key component of clear vision is sufficient light to enable the surgeon to see tissue, distinguish anatomical structures, and eliminate shadows cast by overhead lights. In order to provide such vision, a surgeon may wear head-mounted lights to provide additional lighting and/or lighting techniques during open procedures. See, U.S. Patent Publication No. 2007/0097702 entitled, “SURGICAL HEADLIGHT,” the entirety of which is hereby incorporated by reference.

Visualization and differentiation of different tissues and anatomical structures can be enhanced by optimizing the color characteristics of the light used to illuminate the open surgical site. While the means currently exist to control and broadcast light of any color by mixing such combinations of light (e.g. red, green, and blue LEDs), setting color intra-operatively by the surgeon or an assistant would be tedious and time-consuming, and as such is not currently the practice within the art. Further, such adjustment during the procedure would not guarantee that optimum color balance is achieved. As such, a method of optimizing the color characteristics of light for open surgical procedures, and a method for providing a simple, intuitive interface for the surgeon or an assistant to use intra-operatively to adjust the light source for the optimum color is needed within the industry.

### BRIEF DESCRIPTION OF THE SEVERAL VIEW OF THE DRAWINGS

So that the above recited features and advantages of the present invention can be understood in detail, a more particular description of the invention, may be had by reference to the embodiments thereof that are illustrated in the appended drawings. It is to be noted, however, that the appended drawings illustrate only typical embodiments of the invention and are therefore not to be considered limiting of its scope, for the invention may admit to other equally effective embodiments. One of ordinary skill in the art, provided with the below-referenced drawings, specification and appended claims, would be fully aware and would recognize the utility and inclusion of alternative embodiments and structural components.

FIG. 1A is a perspective view of one embodiment of a surgical illumination device constructed in accordance with the present invention and shown on the head of a user.

FIG. 1B is a perspective view of another embodiment of a surgical illumination device constructed in accordance with the present invention.

FIG. 2 is a schematic block diagram of one embodiment of a surgical illumination device having multiple light sources.

FIG. 2A is a schematic block diagram of an alternate embodiment of a surgical illumination device having multiple light sources.

FIG. 3 is a schematic block diagram of one embodiment of a tunable light controller for use in the surgical illumination device of FIG. 2 or FIG. 2A.

FIG. 4 is a schematic block diagram of another embodiment of a tunable light controller for use in the surgical illumination device of FIG. 2 or FIG. 2A

FIG. 5 is a schematic block diagram of another embodiment of a tunable light controller for use in the surgical illumination device of FIG. 2 or FIG. 2A.

FIG. 6 is a perspective view of an exemplary tuning device for using in the tunable light controller of FIG. 3, 4, or 5.

FIG. 7 is a diagram of one exemplary method of using the tunable light controller of FIG. 3, 4, or 5.

FIG. 8 is a diagram of another exemplary method of using the tunable light controller of FIG. 3, 4, or 5.

FIG. 9 is a diagram of one exemplary method of providing a database for using in the tunable light controller of FIG. 3, 4, or 5.

### DETAILED DESCRIPTION OF THE EMBODIMENTS

Present embodiments of the invention are shown in the above-identified figures and described in detail below. In describing the embodiments, like or identical reference numerals are used to identify common or similar elements. The figures are not necessarily to scale and certain features in certain views of the figures may be shown exaggerated in scale or in schematic in the interest of clarity and conciseness.

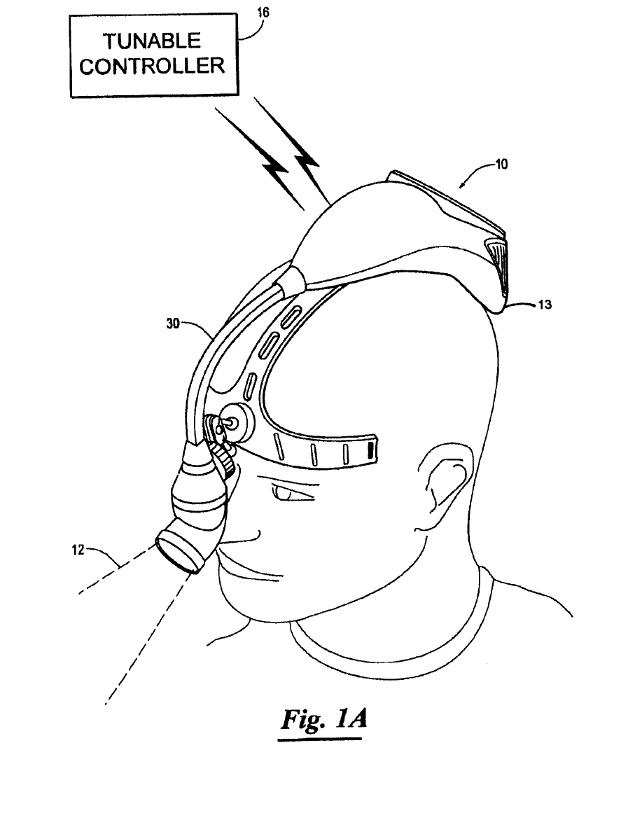

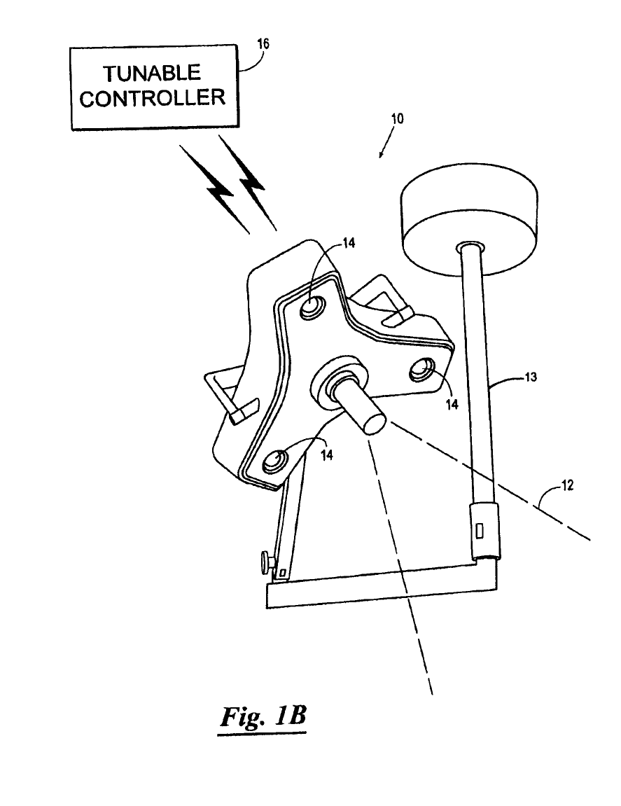

Referring now to the drawings, and in particular to FIGS. 1A and 1B, shown therein and designated by reference numeral 10 is a surgical illumination device 10 for illuminating a surgical field 12 to provide enhanced visual perception for a surgical procedure. The surgical illumination device 10 may be head-mounted (FIG. 1A), ceiling/wall mounted (FIG. 1B), or a stand alone device such as an endoscope, a handheld device or other apparatus that may be either stationary or movable in one or more spatial dimensions.

In general, the surgical illumination device 10 includes one or more light sources 14 selectively activated by a tunable light controller 16 to provide varying wavelengths of light. The tunable light controller 16 controls the light source 14 such that visible or non-visible wavelengths of light are optimized for transmissive and reflective, or functional characteristics of tissue and/or anatomical structures displayed within the surgical field 12. Such adjustment provides enhanced visual perception of the tissue and/or anatomical structures, as well as optionally providing visible or non-visible, yet functional effects to the tissue and/or anatomical structures within the surgical field 12.

Specifically, visual contrast with adjacent tissues and anatomical structures can be improved. Transmissivity of light through certain tissues, as well as reflectance from tissues underneath differing tissues and structure, can be improved to help locate specific tissues and structures during an open procedure. For example, hemoglobin strongly reflects wavelengths at about 460 nm. Thus, the tunable light controller 16 is programmed to tune the light source 14 to substantially 460 nm when a medical procedure step includes the need to identify hemoglobin, a component of blood. Tuning the light source 14 to substantially 460 nm can help locate arteries that are buried in fatty tissue.

Additionally, the tunable light controller 16 may control the light source(s) 14 such that a particular wavelength of light is produced that is capable of biologically interacting with the tissue, anatomical structures and/or microorganisms within the surgical field 12. For example, but not to be construed as limiting, two common strains of methicillin-resistant Staphylococcus Aureus, commonly known as MRSA, can be substantially eradicated by exposure to blue light having a wavelength of from about 405 nm to about 470 nm and, more particularly, 405 nm and 470 nm. At the 470 nm wavelength, the blue light does not emit ultraviolet radiation and may be preferred. In such a manner, the tunable light controller 16 photo-irradiates the surgical field 12 with the desired wavelength of light and thereby significantly decreases the incidence of MRSA. Such photo irradiation can be delivered either cutaneously or subcutaneously. See, for example, Enwemeka et al., “Blue 470-nm Light Kills Methicillin-Resistant Staphylococcus Aureus (MRSA) in vitro,” Photomedicine and Laser Surgery, 2009, the entire contents of which are expressly incorporated herein by reference in their entirety. One of ordinary skill in the art would appreciate that the particular wavelength of light chosen is a function of the biological functionality desired and, as such, all known wavelengths of light that are capable of biologically interacting with items of interest within the surgical field 12 are intended to be encompassed within the appended claims directed to the use of the tunable light controller 16.

The tunable light controller 16 may also control the light source(s) 14 such that a particular wavelength of light is produced that is capable of functionally interacting with one or more organic and/or inorganic compounds present within the surgical field 12. For example, nanoparticles and/or quantum dots can be illuminated in vitro by the wavelengths of light produced by the tunable light controller 16. In one example, nanomaterials, such as nanoparticles and quantum dots, will selectively migrate to the site of a tumor or other targeted diseased or infected state within a host. The tunable light controller 16 can be used to control the light source(s) 14 to selectively illuminate the nanomaterials with specific and/or predetermined wavelengths of light. The tunable light controller 16 can also be used to activate nanomaterials that have been conjugated to drugs and/or other therapeutic agents in order to release the drugs (or therapeutic agent) or to perform some additional biologically active transformations or processes—e.g., luminescing nanoparticles that luminesce under exposure to wavelengths of light directed by the tunable light controller 16 can reveal tumors too tiny to detect by other means or allow a surgeon to be sure all of a cancerous growth has been removed.

The tunable light controller 16 may also be utilized to control the light source(s) 14 to facilitate the illumination of fluorescent dies, labels or markers, bioluminescent materials, or image contrast labels in an in vivo application. In this application, the surgical illumination device 10 takes advantage of the inherent absorption and reflection characteristics of various tissues or the like to show contrast and/or the use of other natural and/or man-made materials to enhance the contrast.

As used herein, the surgical field 12 refers to the region of interest in open surgical procedures such as cardiothoracic, neurosurgery, orthopedic surgery, and the like. It should be noted the surgical field 12 may also refer to the region of interest in endoscopic procedures, dental procedures, human/animal diagnostics, and the like. Additionally, although the term surgical field 12 is used, the surgical illumination device 10 may be used outside the medical field in other areas such as gemology, geology, ocean research, and other fields that could be aided with the use of tunable light in accordance with the present invention. In particular, the surgical illumination device 10 may be used to enhance the safety of food products entering into the food chain by providing the means to irradiate the food products with specific preselected wavelengths of light, either singly or in combinations of wavelengths of light, in order to eradicate the presence of bacteria and/or microorganisms on or within the food products. The surgical illumination device 10 could also be used as a hygienic device for enforcing safety measures (e.g., sterilization protocols in hospital and/or manufacturing circumstances) either in a broad based manner—i.e., entire floors, rooms, equipment etc.—or in a user specific manner whereby the user must place their hands, feet or other appendages into the wavelengths of light provided by the surgical illumination device 10.

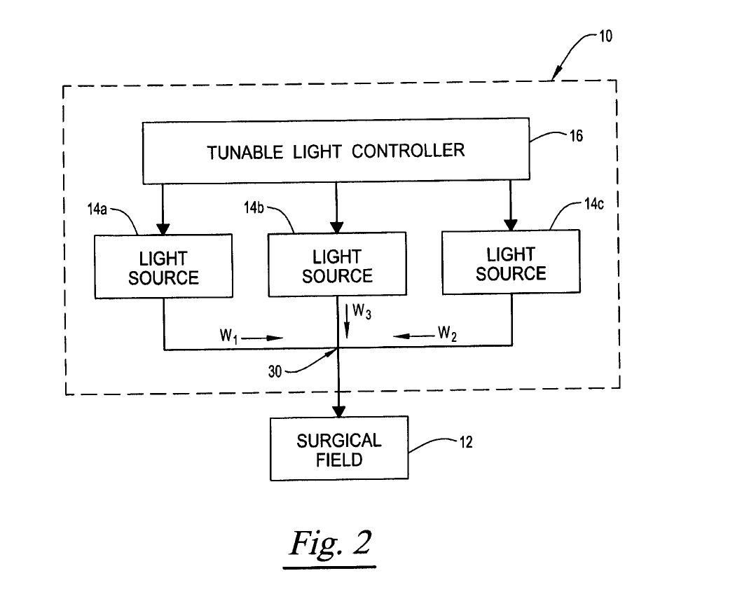

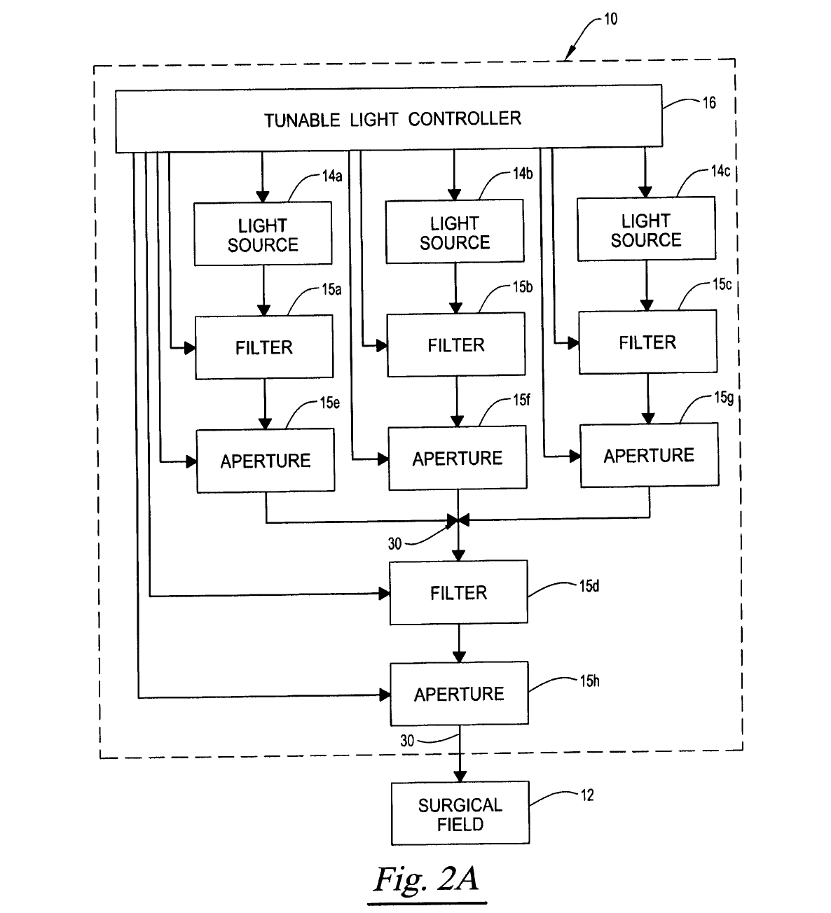

FIG. 2 provides a block diagram illustrating one embodiment of the surgical illumination device 10 having a surgical light 13 using three different light sources, 14a, 14b, and 14c. The light sources 14a, 14b, and 14c within the surgical light 13 may include lasers, LED's, or the like. In the preferred embodiment, the light sources 14a, 14b, and 14c are red, green, and blue LEDs. Other light sources may be used as long as they generate light of specific spectra that can be mixed together to create white or other specific colors. For simplicity, the following description illustrates the use of three light sources 14a, 14b, and 14c; however, it will be apparent to one skilled in the art that a single light source or multiple lights sources (e.g. one red LED, two green LEDs, one blue LED) may be used in accordance with the present invention.

Each of the light sources 14a, 14b, and 14c provides a specific wavelength or wavelength range and combine to provide optimized light for transmissive and reflective characteristics of tissue and/or anatomical structures displayed within the surgical field 12. In one embodiment, as illustrated in FIG. 1A, the wavelengths may be combined and then passed through an optical waveguide 30. The optical waveguide 30 functions as an integrating rod, and guides and combines the wavelengths from each of the light sources 14a, 14b, and 14c to the surgical field 12. Alternatively, as illustrated in FIG. 1B, the wavelengths may be combined within the surgical field 12 or by using an optical scrambler.

Combining the light sources 14a, 14b, and/or 14c allows for a multitude of wavelength combinations. For example, the three separate light sources 14a, 14b, and 14c, providing three separate wavelengths (W₁, W₂, W₃) respectively, allow for a multitude of combinations between the three wavelengths (i.e. W₁×W₂, W₂×W₃ . . . ) Alternatively, each light source 14a, 14b, and 14c may provide for multiple wavelengths. For example, light source 14a may alone provide three separate wavelengths (W_{1A}, W_{2A}, W_{3A}). The multiple combinations of wavelengths of light enable the enhanced visualization of tissues and anatomic structures by improving contrast, transmissivity, and reflectivity.

Additionally, the intensity of light within the surgical field 12 may be manipulated by altering the projection of light from each light source 14a, 14b, or 14c, altering the projection of light from the optical waveguide 30, or a combination of both. For example, altering the amplitude of the wavelength of light projected by the light source 14a can vary the intensity and relative brightness perceived by the surgeon in the surgical field 12. For example, shown in FIG. 2A is a schematic diagram of an alternative embodiment of the surgical illumination device 10. In the embodiment shown in FIG. 2A, the surgical illumination device 10 is provided with additional light tuning apparatus for controlling the amplitude and the wavelengths of light projected by the surgical illumination device 10, such as to eliminate undesirable wavelengths in particular specific applications. In the example shown in FIG. 2A the light tuning apparatus are shown as optical filters 15a, 15b, 15c and 15d; as well as controllable apertures 15e, 15f, 15g and 15h. The optical filters 15a, 15b, 15c and 15d can be any suitable type of controllable optical filters, such as dichromatic filters or a geometric mixing device, such as a prism where the angle of incidence of light entering the prism is controlled to change the optical properties of the light exiting the prism. The controllable apertures 15e, 15f, 15g and 15h can be implemented in any suitable fashion, such as by using mechanical or liquid crystal shutters or the like.

The optical filters 15a, 15b, 15c and 15d and apertures 15e, 15f, 15g and 15h can be located anywhere within the path of the light being generated by the light source(s) 14 and serve to control the passage of light. For example, the optical filters 15a, 15b and 15c can be located prior to the combiner 30 so that the optical filters 15a, 15b and 15c control the amplitude and/or wavelength of the light being generated for specific ones of the light sources 14a, 14b and 14c; while the optical filter 15d is located after the combiner 30 to control aspects of the combined light. The apertures 15e-h can be adjusted to increase or decrease radiant energy in the field of view, i.e., the surgical field 12. Some wavelengths of light may be brighter than others and require a larger aperture to provide the desired contrast. Other wavelengths may be too bright and require some limitation of illumination energy.

Generally, each surgical illumination device 10 has the ability to provide ranges of intensity and color of light within a predefined range. Activation of the light sources 14 by the tunable light controller 16 provides pre-programmed color settings that enhances visualization of tissues and anatomic structures within the surgical field 12 by improving contrast, transmissivity and reflectivity. The pre-programmed color settings provide a mechanism to provide enhanced visual perception at each medical procedure step.

Further, the surgical illumination device 10 can be used for the destruction of pathogens or unwanted tissue in vivo through continuous, manual (on-demand), or automated manipulation of the light output to increase of specific wavelengths known to destroy those pathogens. The wavelengths used to destroy the pathogens or unwanted tissue can be delivered through the same optics as the other wavelengths discussed herein or through additional optical paths with light source(s) 14 tuned to the desired pre-determined wavelength.

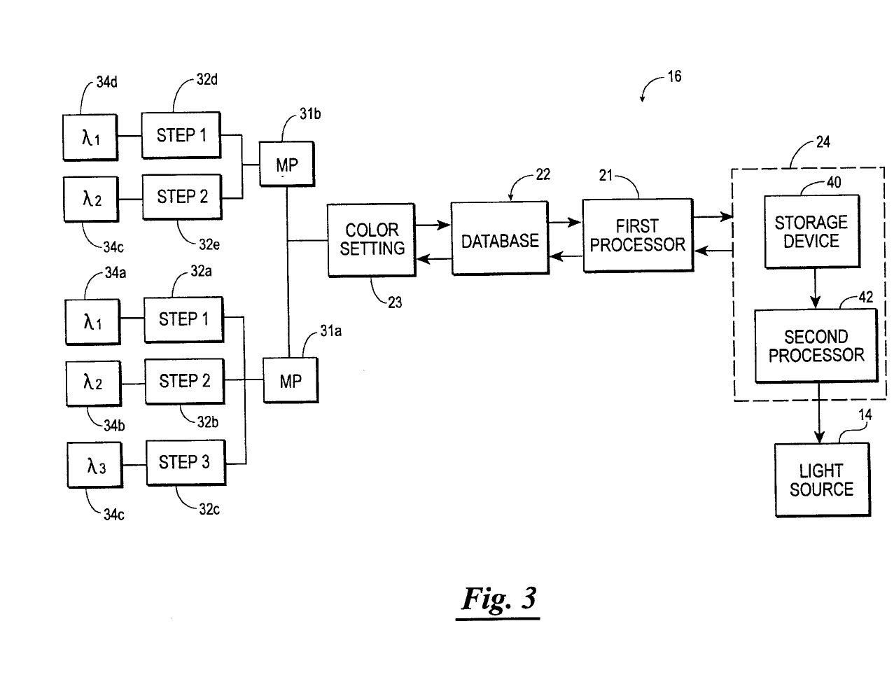

FIG. 3 is an exemplary hardware diagram for the tunable light controller 16. Generally, the tunable light controller 16 is a single system or multiple systems that are able to embody and/or execute the logic of the processes described herein. The logic embodied may be executed on any appropriate hardware such as, for example, a dedicated system or systems, a microcontroller, field-programmable gate array, application-specific integrated circuit, personal computer system, distributed processing computer system, and/or the like. The hardware and software used are designed with two key concerns: flexibility and scalability. Although specific software and hardware components are described herein, it will be understood that a wide array of different components may be substituted.

The tunable light controller 16 is programmed to implement some or all of the methods of the present invention, as will be described in more detail below. In general, the tunable light controller 16 includes a first processor 21 communicating with a database 22. The first processor 21 retrieves pre-programmed color settings 23 for one or more medical procedures from the database 22 and provides the pre-programmed color settings 23 for the specific medical procedure to at least one tuning device 24. Using the pre-programmed color settings 23 for the specific medical procedure retrieved from the database 22, the tuning device 24 controls the light source 14 to provide enhanced visual perception of the surgical field 12 or additional functionality for the medical procedure. The database 22 can be a traditional database organized by files, records and fields, or a hypertext database having links between objects, or other suitable type of database. The database 22 is preferably stored on one or more computer readable medium and is hosted and/or executed by a computer which may be the same or different from the first processor 21. The database 22 includes a database management system to permit user(s) to enter, organize, locate and/or select data in the database.

The database 22 can be organized by specific application and stores an identification of the specific application and at least one pre-programmed color setting for the specific application. Examples of specific applications include medical procedures, tissue types or anatomical structures. The database 22 will be described hereinafter by way of example with the specific application being a medical procedure. The database can be organized by medical procedure and stores an identification of the medical procedure and at least one pre-programmed color setting 23 for each medical procedure or medical procedure step. The preprogrammed color setting 23 is adapted to facilitate a first assigned illumination by the surgical light. The pre-programmed color setting 23 includes at least one medical procedure 31. For example in FIG. 3, the pre-programmed color setting 23 includes medical procedures 31a and 31b. Each medical procedure 31a and 31b include one or more medical procedure steps with each medical procedure step having an assigned illumination. In this regard, medical procedure 31a includes three medical procedure steps 32a, 32b, and 32c. Each medical procedure step 32a, 32b, and 32c includes an assigned illumination 34a, 34b, and 34c. The assigned illumination 34a, 34b, and 34c is that wavelength, intensity and/or combinations thereof, of light for the medical procedure step that adjusts the physical properties and/or characteristics of the light to provide enhanced visual perception in the surgical field 12. Particular filtering characteristics or aperture settings can also be stored in the database 22.

In the same regard, medical procedure 31b includes two medical procedure steps 32d and 32e. Each medical procedure step 32d and 32e includes an assigned illumination 34d and 34e. The assigned illumination 34d and/or 34e is that wavelength, intensity, and/or combination thereof of light for the medical procedure step that adjusts the physical properties and/or characteristics of light to provide enhanced visual perception in the surgical field 12. For example, in a CABG (coronary artery bypass graft), the left anterior descending coronary artery can either be hidden in fat, or in heart muscle. Optimizing the light source 14 can help the surgeon quickly discover if this artery is in fat, or if not, help him find it for dissection in muscle tissue. This can save critical time during surgery while the patient is on heart bypass. Optimizing the light source 14 to save critical time during surgery can help in the nearly 500,000 CABG procedures performed annually in the U.S.

Optionally, the database 22 stores pre-programmed color settings for the different tissues as well as the different dies, labels and markers and combinations thereof, for optimal visualization of each.

The assigned illuminations 34a-34e can be determined in a variety of manners. For example, the assigned illuminations 34a-34e may be determined by: 1) wavelengths of light found in research literature associated with tissue and/or anatomical structures; 2) pre-programmed color settings provided by the surgical illumination device 10 through research; 3) minute adjustments of baseline setting provided by the user during simulated or actual surgical procedures, and/or 4) scanning available illumination to provide the assigned illuminations. Once the assigned illuminations 34a-34e are determined for particular medical procedures or particular medical procedure steps, such assigned illuminations are programmed or stored in the database 22. It should be noted that although the term “illumination” is used, assigned illuminations 34a-34e may include a suitable illumination range. For example, the surgeon may adjust the wavelengths of each color over a permitted range, to suit his individual preference, or to simply adjust a “Warmer/Cooler” control to change to overall color of the light by a slight amount.

Research literature, such as A. Edward Profio's article in Applied Optics entitled, “Light transport in tissue,” Applied Optics, Vol. 28, Issue 12, (June 1989), provide data of specific light frequencies at which tissue and/or anatomical structures have enhanced visual perception. See Profio, A. Edward. “Light transport in tissue.” Applied Optics 28:12 (June 1989): pp. 2216-2221. The database 22 may include precise wavelengths, such as described in the above referenced journal article, as the assigned illuminations 34a-34e and/or substantially similar values for association with each medical procedure step 32a-32e.

Additionally, the assigned illuminations 34a-34e may be determined by research or trial and error techniques using the surgical illumination device 10. For example, the surgical illumination device 10 may be programmed to provide a mode of operation that allows tuning to any wavelength of light within a predefined range. In this mode of operation, the user is able to tune the light to any particular wavelength of light that provides enhanced visual perception for the specific tissue and/or anatomical structure during the medical procedure. This wavelength value can then be saved within the database 22 for future use.

The user may also provide assigned illuminations of light using prior assigned illuminations. For example, in the medical procedure 31a the medical procedure step 32a includes the assigned illumination 34a. This assigned illumination 34a becomes a baseline color output. Using the tuning device 24, the user can minutely adjust the baseline color output to a particular wavelength of light that provides enhanced visual perception for the specific tissue and/or anatomical structure during the medical procedure step 32a. This specific wavelength of light may be stored as the assigned illumination 34a or as an alternate assigned illumination for medical procedure step 32a for future use.

The minute adjustments made to the assigned illumination 34a are also capable of being stored for use in, not only the medical procedure step 32a of medical procedure 31a, but also other medical procedure steps and/or medical procedures. For example, the minute adjustments may be common adjustments made to the illuminations based on the surgeon's particular needs and desires. These common adjustments may be used in multiple procedures. As such, saving these customized settings allows the surgeon to be able to quickly and efficiently set the assigned illumination 34a to their own particular needs and desires independent of the particular medical procedure step and/or medical procedure.

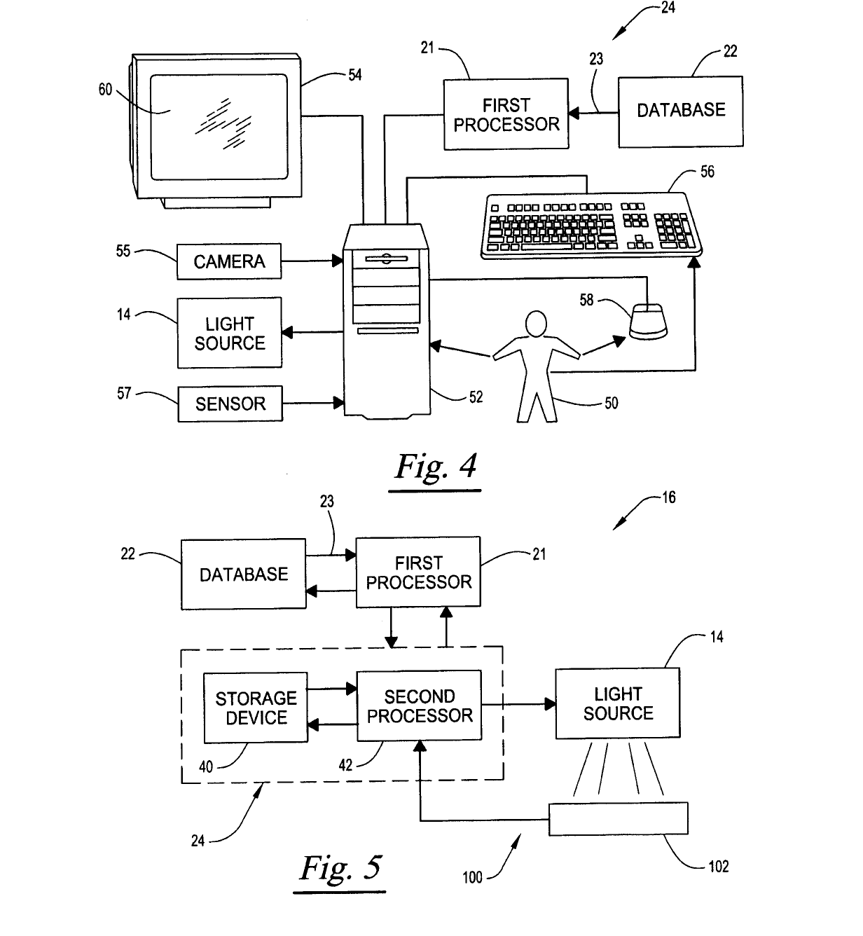

Additionally, the assigned illumination 34a may be determined by scanning through all available illuminations. For example, the tuning device 24 may provide a scanning function that allows manual and/or automatic progression through all available illuminations, or between two limits that define a series of wavelengths. The user may instruct the tuning device 24 vocally and/or manually to stop on a particular wavelength. Alternatively, a sensor 57 (shown in FIG. 4) may be placed in or near the surgical field 12 to automatically determine the optimal wavelength for illumination forming a feedback loop with the tuning device 24. The sensor(s) 57 detect and measure the reflected energy. The signals produced by the sensor(s) 57 can be used to adjust the color output based on in vivo readings. The sensor(s) 57 can also be used for in vitro procedures. This wavelength may override the assigned illumination and/or represent the assigned illumination for the medical procedure step.

The first processor 21 is capable of retrieving one or more medical procedures 31 as pre-programmed color settings 23 from the database 22 and providing this data to the tuning device 24. The configuration of the first processor 21 will depend greatly upon requirements and needs of the particular embodiment of the tunable light controller 16. As one skilled in the art will appreciate, the first processor 21 may include a logic based system, such as a microprocessor, field programmable gate array, digital signal processor, and/or microcontroller capable of executing instructions for retrieving data from the database 22 and providing the data to the tuning device 24. For example, the first processor 21 may be a personal computer containing an internal database 22. Alternatively, the first processor 21 may include multiple logic based systems capable of providing the data to the tuning device 24.

It is contemplated that one or more elements of the first processor 21 and/or tuning device 24 will be enabled to individually run software and the like in order to implement the methods of the present invention. In this regard, the tuning device 24 need not be in communication with the first processor 21 and, instead, may be periodically connected and/or placed in communication with the first processor 21 so as to synchronize and/or transfer all, or a portion of, the pre-programmed color settings 23 stored on the first processor 21 and/or tuning device 24. For example, the first processor 21 may be connected to the tuning device 24 to upload the pre-programmed color settings 23 to the tuning device 24, and then disconnected from the tuning device 24.

In another embodiment, the first processor 21 connects with the tuning device 24 over a network to provide the pre-programmed color setting 23. The network can be an intranet, the Internet, or any other network as will be appreciated by one skilled in the art. The preferred embodiment of the network exists in an Internet environment, meaning a TCP/IP-based network. However, it is conceivable that in the near future it may be advantageous for the preferred or other embodiments to utilize more advanced networking technologies. In addition, the network does not refer only to computer-based networks, but can also represent telephone communications, cable communications, and similar networking technologies.

In one embodiment, as illustrated in FIG. 3, the tuning device 24 includes a storage device 40 and a second processor 42. The tuning device 24 receives the pre-programmed color settings 23 from the first processor 21 and stores all, or a portion of, the data in the storage device 40. The second processor 42 retrieves the data from the storage device 40, and using this data, controls the light source 14 providing enhanced visual perception for each step of the medical procedure.

The storage device 40 may include storage media such as a smart card, SIM card, flash drive, and/or the like. In the preferred embodiment, the storage device 40 is periodically connected and/or placed in communication with the first processor 21 so as to synchronize and/or transfer all, or a portion of, the pre-programmed color settings 23 provided by the first processor 21.

The second processor 42 uses all, or a portion of, the pre-programmed color settings 23 stored on the storage device 40 to provide enhanced visual perception of tissues and/or anatomical structures in the surgical field 12. The second processor 42 may include integral pulse-width modulation circuitry or other similar mechanisms to drive the light source 14, or it may generate control outputs to separate discrete pulse width modulation circuitry to individually control each of the light sources. Furthermore, processor 42 may include feedback circuitry to measure the color and light amplitude of each source to provide closed-loop feedback control of the output of each light source.

As previously discussed, each pre-programmed color setting 23 may be assigned to more than one medical procedure step, and each medical procedure step may have a different assigned illumination. For example, medical procedure 31a includes three medical procedure steps 32a-32c with assigned illuminations 34a-34c. An input device, such as a microphone, mechanical switch, button, keypad, touch screen, timer, sensor, or the like, may be used to provide input signals to the second processor 42 to cause the second processor 42 to cycle through each medical procedure step 32a-32c. The switch may be mechanical, electrical, and/or the like. For example, the switch may be a push-button switch. When the input device is implemented as a microphone, the second processor 42 may be programmed with voice recognition capabilities to respond to a user's verbal command.

Visual and audio feedback may also be optionally provided by the second processor 42 to allow for confirmation of the change from each medical procedure step. For example, during medical procedure 31a, the assigned illumination 34a will switch to assigned illumination 34b after medical procedure step 32a is complete. At that time, an LED or LCD indicator light can provide visual confirmation that the assigned illumination has changed from 34a to 34b, or is about to change from 34a to 34b. This indicator may also indicate the previous, current, and next step in the surgical procedure.

FIG. 4 illustrates another embodiment of the tuning device 24. In this embodiment, the tuning device 24 involves a user 50 using a computer 52, with a monitor 54, one or more camera(s) 55, a keyboard 56, one or more sensor(s) 57 and a mouse 58. The user 50 may use software 60 to render content received from the first processor 21. For example, the software 60 may include a “browser” providing content to the user 50. In this regard, the tuning device 24 obtains the pre-programmed color settings 23 from the first processor 21 through the use of the “browser” and controls the light source 14 using all, or a portion of, the data. Communication between the tuning device 24 and the light source 14 may be wired or wireless. The one or more camera(s) 55 can be directed at the surgical field 12 to generate images (or video) of one or more portions of the surgical field 12 during a procedure so that such images and/or video can be analyzed and/or displayed on the monitor 54. The software 60 may include an image analysis module that when executed causes the computer 52 to analyze the images in real-time during the procedure to transform the images or cause additional information regarding the procedure to be displayed by the monitor 54. For example, the image analysis module may include object recognition techniques to cause the computer 52 to locate particular objects, such as particular tissues, pathogens, lesions or tumors, within the surgical field 12 and to notify the surgeon of the existence of such objects. For example, located objects can be displayed on the monitor 54 in bold or in a different color from the remainder of the image and/or video. Image capture could be enhanced with visualization techniques such as differential interference contrast and used to continually improve the database. Visual enhancement techniques would increase the reliability of the identification of different tissues or pathogens. Incorporating automated digital pathology recognition into the database could highlight known pathogens and alert the users of the presence of pathogens that they may not have known were present.

The one or more sensor(s) 57, such as radiometers, photodiodes, phototransistors or the like can be used to detect and measure the reflected energy from the surgical field 12. The computer 52 can use the signals generated by the sensor(s) 57 to adjust the color output based on in vivo readings. The one or more sensor(s) 57 can also be used for in vitro procedures.

The tuning device 24 may be designed to provide flexibility in its deployment. Depending upon the requirements of the particular embodiment, the tuning device 24 may be designed to work in almost any computing environment such as a desktop application, a web application, a series of web services designed to communicate with an external application, and/or the like.

The tuning device 24 may also be implemented as a portable device 62. Examples of the portable device 62 include, but are not limited to, a laptop computer, cellular telephone, a PDA, or other type of device capable of requesting and receiving content from the first processor 21 and controlling the light source 14 to provide enhanced visual perception at each step of the medical procedure.

Other methods and/or steps described herein may be implemented through software enabling a surgeon and/or researcher to adapt the tunable light controller 16 to implement such methods and/or steps. For example, software may comprise instructions for such methods and/or steps, with such instructions stored on one or more computer-readable media. Computer-readable media may include, for example, diskettes, compact discs (CDs), digital video discs (DVDs), flash drives, servers, hard drives, and/or the like. Such software may be distributed in any suitable fashion such as by providing the surgeon/researcher with software or permitting the surgeon/researcher to download the software.

Referring now to FIG. 5, shown therein is an embodiment of the tunable light controller 16 including a control system 100 to adjust, regulate, and/or further control the resulting light in the surgical field provided by the pre-programmed color settings 23. By way of example, the control system 100 will be described for controlling the light source 14. However, it should be understood that the following description is equally applicable to control elements of the tunable light controller 16 such as the first processor 21 and/or second processor 42.

The control system 100 includes a feedback mechanism 102 in communication with the second processor 42. The feedback mechanism 102 includes the sensor(s) 57 which detect and generate signals indicative of the actual physical aspects (e.g., color, intensity or the like) related to the light promulgating from the light source 14. Preferably, the feedback mechanism 102 functions automatically, i.e. without any human intervention. The second processor 42 receives signals from the feedback mechanism 102 indicative of one or more physical aspects related to the light promulgating from the light source 14, and then utilizes such signals to further alter and/or control the light source 14. For example, the feedback mechanism 102 can determine whether the first assigned illumination has been achieved using the computer 52 and the sensor(s) 57 by comparing the actual physical aspects with the first assigned illumination or data indicative thereof. The feedback mechanism 102 can also include a visual indicator, such as the monitor 54, to output a signal that the first assigned illumination has or has not been achieved.

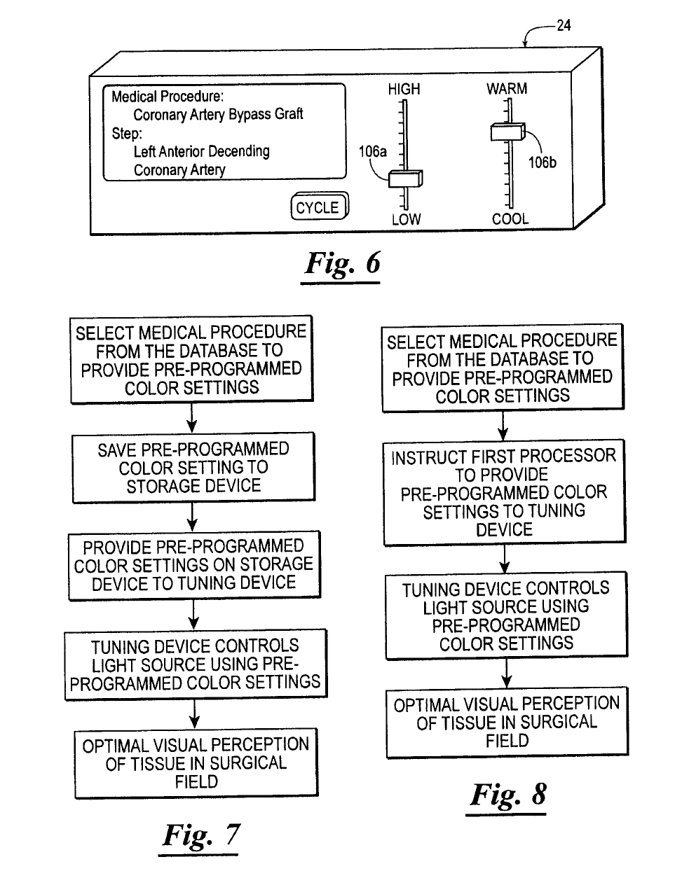

The feedback mechanism 102 may also be user operated. In this regard, the control system 100 can further adjust the pre-programmed color settings 23 to an individual's preference. The feedback mechanism 102 provides user-operated control to adjust minutely the pre-programmed color settings 23 to individual preferences. FIG. 6 illustrates one embodiment of the feedback mechanism 102 having linear sliders 106a and 106b. Linear slider 106a adjusts the intensity of the light output from the pre-programmed color settings 23 based on a sliding scale. Linear slider 106b adjusts the color perception from the pre-programmed color settings 23 based on color theory. For example, a user can adjust the color perception based on a “warmer” or “cooler” color preference. User control of this feedback mechanism 102 may also be implemented using other hardware controls, such as rotary knobs or joysticks, a touch screen, audible (voice) input, or any other user input method, as will be appreciated by one skilled in the art. This may be the same display and input method used for selecting the particular step of the surgical procedure, as selected through software internal to the device, or it may be an entirely separate indicator and input method(s) described previously. Also, the surgeon may “fine tune” the optimum color setting using the feedback mechanism described previously.

The following examples of methods for using the tunable light controller 16 are set forth hereinafter. It is to be understood that the examples are for illustrative purposes only and are not to be construed as limiting the scope of the invention as described.

### Example 1

Referring to FIG. 7, using the first processor 21, a user selects one or more medical procedures 31 to provide pre-programmed color settings 23 from the database 22. The user instructs the first processor 21 to save the medical procedures on the storage device 40. The user then provides the storage device 40 to the tuning device 24. The tuning device 24 retrieves the pre-programmed color settings 23 from the storage device 40, and using this data, controls the light source 14 providing enhanced visual perception of tissue and/or anatomical structures in the surgical field 12 for each medical procedure step 32. The surgeon can select or cycle through each optimized illumination setting that corresponds to the particular step in the surgical procedure using the methods described above. In addition, the surgeon can adjust or “fine tune” the optimum illumination using the user controlled feedback mechanism described above.

### Example 2

Referring to FIG. 8, using the first processor 21, a user selects one or more medical procedures to provide pre-programmed color settings 23 from the database 22. The user instructs the first processor 21 to provide the medical procedure to the tuning device 24 through a network connection such as a wireless TCP/IP-based network. The tuning device 24 controls the light source 14 based on the pre-programmed color settings 23 for the medical procedure providing enhanced visual perception. The surgeon can select or cycle through each optimized illumination setting that corresponds to the particular step in the surgical procedure using the methods described above. In addition, the surgeon can adjust or “fine tune” the optimum illumination using the user controlled feedback mechanism described above.

### Example 3

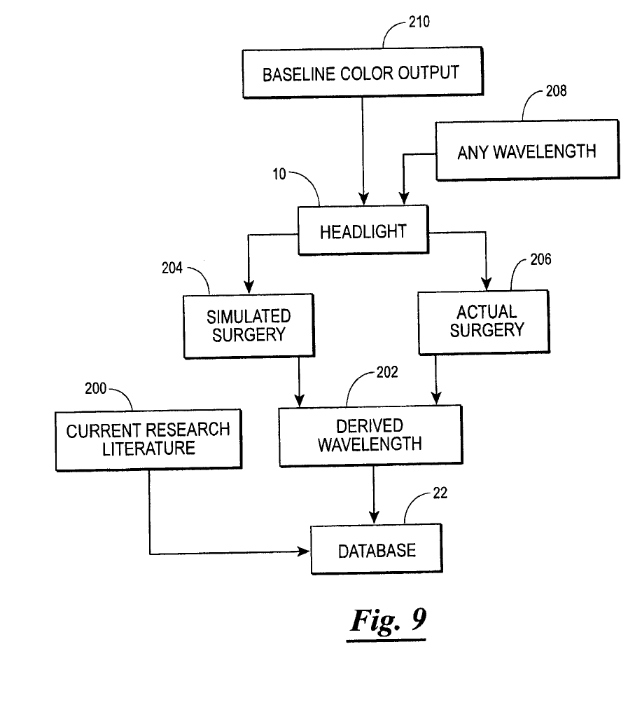

FIG. 9 is a block diagram illustrating several methods of providing medical procedures to the database 22. For example, the database 22 may be provided with current research literature 200 available to the general public regarding enhanced light frequency output for visualizing tissue and/or anatomical structures.

Additionally, the database may include derived wavelengths 202 provided by the surgical illumination device 10. For example, the surgical illumination device 10 may be tunable to any and all wavelengths 208 of light. During simulated surgery 204, or actual surgery 206, the surgical illumination device 10 may be tuned to the derived wavelength 202. The derived wavelength 202 being the wavelength that illuminates and differentiates the tissue and/or anatomical structure of interest. The derived wavelength 202 may include a wavelength range. This derived wavelength range may be saved into the database 22 for subsequent use.

The database 22 may also include derived wavelengths 202 that are minutely adjusted baseline color outputs 210. For example, the user may be provided with the baseline color output 210. The baseline color output 210 can then be further minutely adjusted during simulated surgery 204, or actual surgery 206, to provide the light frequency output to be saved into the database 22.

The foregoing method of providing optimum illumination data for surgical procedures may be implemented using computer software that includes a human interface (screen display) that shows the location of the color on, for example, a CIE 1931 chromaticity diagram, or similar diagram, along with an indication of the relative intensity of each of the light sources. This interface may also include step-by-step instructions to the user to enable them to optimally tune the illumination output.

The foregoing disclosure includes the best mode for practicing the invention. It is apparent, however, that those skilled in the relevant art will recognize variations of the invention that are not described herein. While the invention is defined by the appended claims, the invention is not limited to the literal meaning of the claims, but also includes these variations.

What is claimed:

1. A surgical illumination device for illuminating a surgical field to provide enhanced visual perception of a tissue during a medical procedure, comprising:
a surgical light comprising:
a first light source providing a first wavelength of light;
a second light source providing a second wavelength of light;
a combiner receiving and combining the first and second wavelengths of light; and
a tunable light controller comprising:
a database organized by medical procedure and storing an identification of the medical procedure and at least one pre-programmed color setting for each medical procedure, the preprogrammed color setting adapted to facilitate a first assigned illumination;
a tuning device receiving the pre-programmed color setting stored in the database and controlling the first light source and the second light source such that the first wavelength and the second wavelength combine to provide the first assigned illumination.

2. The surgical illumination device of claim 1, wherein the combiner comprises an optical waveguide receiving the wavelengths of light from the first and second light sources to guide and combine the wavelengths of light.

3. The surgical illumination device of claim 1, wherein at least one medical procedure in the database includes at least two medical procedure steps with each medical procedure step having at least one pre-programmed color setting adapted to be used by the tuning device to control the first light source and the second light source to provide an assigned illumination for each step of the medical procedure.

4. The surgical illumination device of claim 3, wherein the tuning device includes a processor executing a scanning function that allows progression through the medical procedure steps.

5. The surgical illumination device of claim 1, wherein the tuning device includes a scanning function that allows progression through all available illuminations.

6. The surgical illumination device of claim 1, wherein the tuning device includes a computer programmed with a web browser to retrieve the pre-programmed color setting from the database.

7. The surgical illumination device of claim 1, further comprising a control system including a feedback mechanism detecting and passing signals indicative of one or more physical aspects of the light promulgating from the light source to the tuning device, and wherein the tuning device utilizes such signals to alter and/or control the light source.

8. The surgical illumination device of claim 7, wherein the tuning device includes an input device manipulated by a user to adjust a pre-programmed color setting.

9. The surgical illumination device of claim 1, wherein the surgical illumination device further comprises means for determining whether the first assigned illumination has been achieved, and a visual indicator outputting a signal that the first assigned illumination has been achieved.

10. The surgical illumination device of claim 1, wherein the tuning device further comprises a computer with a monitor, a storage device and a camera, the camera being directed at the surgical field to generate an image of the surgical field and to provide the images to the computer, the storage device storing software including an image analysis module that when executed by the computer causes the computer to analyze the image in real-time during the procedure, and cause additional information regarding the image to be displayed on the monitor.

11. The surgical illumination device of claim 10, wherein the image provided to the computer depicts contrasting tissues or materials, and wherein the image analysis module when executed by the computer causes the computer to enhance the contrasting tissues or materials.

12. A method for controlling a surgical light during a predefined medical procedure having at least two medical procedure steps, the surgical light comprising a first light source providing a first wavelength of light, a second light source providing a second wavelength of light, and a combiner receiving and combining the first and second wavelengths of light, the method comprising the steps of:
for the medical procedure steps:
retrieving at least one pre-programmed color setting adapted to provide an assigned illumination for a particular medical procedure step; and
controlling the first light source and the second light source to provide the assigned illumination for the particular medical procedure step.

13. A method for enhancing the control of a surgical light during predefined medical procedures having at least two medical procedure steps, the surgical light comprising a first light source providing a first wavelength of light, a second light source providing a second wavelength of light, and a combiner receiving and combining the first and second wavelengths of light, the method comprising the steps of:
hosting a database organized by medical procedure and storing an identification of the medical procedure and at least one pre-programmed color setting for each medical procedure, the preprogrammed color setting adapted to facilitate a first assigned illumination by the surgical light; and
select a particular medical procedure in the database; and
providing the identification of the medical procedure and the at least one pre-programmed color setting.

14. A surgical illumination device for illuminating a surgical field, comprising:
a surgical light comprising:
a first light source providing a first wavelength of light;
a second light source providing a second wavelength of light;
a combiner receiving and combining the first and second wavelengths of light; and
a tunable light controller comprising:
a database storing identifications of specific applications and at least one pre-programmed color setting for each specific application, the preprogrammed color setting adapted to facilitate a first assigned illumination for the specific application;
a tuning device receiving the pre-programmed color setting from the database and controlling the passage of the first and second wavelengths of light to provide the first assigned illumination.

15. The surgical illumination device of claim 14, wherein the tuning device includes optical filters to control the passage of the first and second wavelengths of light.

16. The surgical illumination device of claim 14, wherein the tuning device includes a controllable aperture to control the passage of the first wavelength of light.

17. The surgical illumination device of claim 14, wherein the combiner comprises an optical waveguide receiving the wavelengths of light from the first and second light sources to guide and combine the wavelengths of light.

18. The surgical illumination device of claim 14, wherein the specific application is a medical procedure, and wherein the database includes at least two medical procedure steps for the medical procedure with each medical procedure step having at least one pre-programmed color setting adapted to be used by the tuning device to provide an assigned illumination for each step of the medical procedure.

19. The surgical illumination device of claim 14, wherein the tuning device includes a processor executing a scanning function that allows progression through the medical identifications of specific applications.

20. The surgical illumination device of claim 18, wherein the tuning device includes a processor executing a scanning function that allows progression through the medical procedure steps.

21. The surgical illumination device of claim 14, wherein the tuning device includes a scanning function that allows progression through all available illuminations.

22. The surgical illumination device of claim 14, wherein the tuning device includes a computer programmed with a web browser to retrieve the pre-programmed color setting from the database.

23. The surgical illumination device of claim 14, further comprising a control system including a feedback mechanism detecting and passing signals indicative of one or more physical aspects of the light promulgating from the light source to the tuning device, and wherein the tuning device utilizes such signals to alter the illumination of the surgical field.

24. The surgical illumination device of claim 23, wherein the tuning device includes an input device manipulated by a user to adjust a pre-programmed color setting.

25. The surgical illumination device of claim 14, further comprising means for determining whether the first assigned illumination has been achieved, and a visual indicator outputting a signal that the first assigned illumination has been achieved.

26. The surgical illumination device of claim 14, wherein the tuning device further comprises a computer with a monitor, a storage device and a camera, the camera being directed at the surgical field to generate an image of the surgical field and to provide the images to the computer, the storage device storing software including an image analysis module that when executed by the computer causes the computer to analyze the image in real-time during the procedure, and cause additional information regarding the image to be displayed on the monitor.

27. The surgical illumination device of claim 26, wherein the image provided to the computer depicts contrasting tissues or materials, and wherein the image analysis module when executed by the computer causes the computer to enhance the contrasting tissues or materials.
